(19) United States
(12) Patent Application Publication
(10) Pub. No.: US 2012/0197102 A1
(43) Pub. Date: Aug. 2, 2012

(54) OPHTHALMIC SURGICAL MICROSCOPE

(75) Inventors: Masaaki HANEBUCHI, Nukata-gun (JP); Michihiro TAKII, Nukata-gun (JP); Masakazu ENDO, Okazaki-shi (JP); Tsutomu SUNADA, Toyohashi-shi (JP)

(73) Assignee: NIDEK CO., LTD., Gamagori-shi (JP)

(21) Appl. No.: 13/358,921

(22) Filed: Jan. 26, 2012

(30) Foreign Application Priority Data
Jan. 27, 2011 (JP) 2011-015779

(51) Int. Cl. A61F 9/007; A61B 3/16; A61B 6/02; A61B 6/00

(52) U.S. Cl. 600/398; 600/476; 600/427

(57) ABSTRACT

An ophthalmic surgical microscope comprises: an observation optical system for observing a patient's eye during surgery; a corneal shape measuring unit for measuring a corneal shape of the patient's eye placed in a surgical position; and a controller configured to output guide information for guiding a surgery with an intraocular lens based on a measurement result obtained by the corneal shape measuring unit to an output device.

### CROSS-REFERENCE TO RELATED APPLICATIONS

This application is based upon and claims the benefit of priority from the prior Japanese Patent Application No. 2011-015779, filed Jan. 27, 2011, the entire contents of which are incorporated herein by reference.

### BACKGROUND

1. Technical Field

The present invention relates to an ophthalmic surgical microscope to be used in ophthalmic surgery.

2. Related Art

As one of intraocular lenses (IOL), recently, a TORIC-IOL designed to correct astigmatism appears on the market. In this case, a surgeon or operator measures an astigmatic axis of a patient's eye (to be operated on) in advance by use of a corneal shape measuring device (see Patent Document 1). The surgeon then applies a first marking in a horizontal axis of the patient's eye by using a special-purpose member and further applies a second marking at a position corresponding to an astigmatic axis of the eye with reference to the first marking, and he/she inserts an IOL in the eye so that the second marking coincides with the axis of the IOL.

However, there is a possibility that a posture of a patient is different between at the time of measuring a corneal shape and at the time of applying markings, and thus the marking could not be applied correctly in the position corresponding to the astigmatic axis. This may cause displacement of an inserting position of the IOL.

For a surgery with an IOL (IOL surgery), a surgeon performs the IOL surgery while looking through a surgical microscope (see Patent Document 2). Patent Document 2 discloses a surgical microscope including an autorefractometer and an autokeratometer.

The microscope of Patent Document 2 is arranged to measure eye refractive power and corneal shape after extraction of a crystalline lens.

### RELATED ART DOCUMENTS

### Patent Documents

Patent Document 1: JP 2003-169778 A

Patent Document 2: JP 5(1993)-111465 A

### DISCLOSURE OF THE INVENTION

### Problems to be Solved by the Invention

The ophthalmic surgical microscope is desired to have a configuration capable of accurately performing the IOL surgery. For example, it is necessary to consider variations in astigmatic axis, viscoelastic substances, etc.

The present invention has a purpose to provide an ophthalmic surgical microscope valuable in performing IOL surgery with high accuracy.

### Means of Solving the Problems

To achieve the above purpose, one aspect of the invention provides an ophthalmic surgical microscope comprising: an observation optical system for observing a patient's eye during surgery; a corneal shape measuring unit for measuring a corneal shape of the patient's eye placed in a surgical position; and a controller configured to output guide information for guiding a surgery with an intraocular lens based on a measurement result obtained by the corneal shape measuring unit to an output device.

### BRIEF DESCRIPTION OF DRAWINGS

FIG. 1 is a schematic diagram to explain a surgical microscope in an embodiment;

FIG. 2 is a schematic optical diagram to explain a second measuring optical system;

FIG. 3 is a diagram showing an anterior segment image obtained through an anterior segment observation system;

FIG. 4 is an example of an observation image observed through eyepiece lenses, showing a state where a microscopic image before incision and an indication image are superimposed one on the other; and

FIG. 5 is an example of an observation image observed through eyepiece lenses, showing a state where a microscopic image after IOL insertion and an indication image are superimposed one on the other.

### MODE FOR CARRYING OUT THE INVENTION

In the following detailed description, for purpose of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

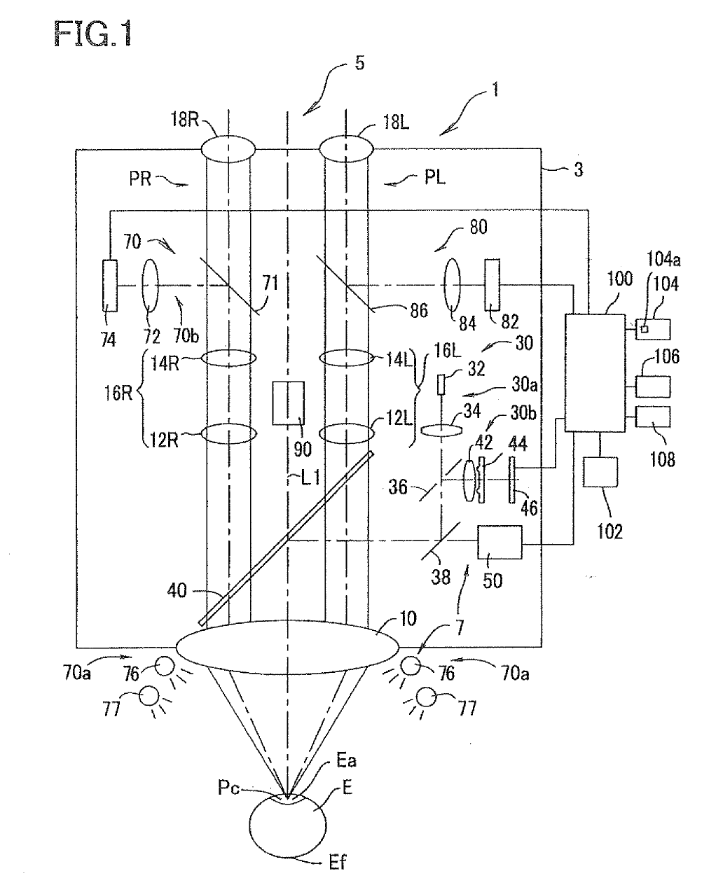

A preferred embodiment of the present invention will be described below referring to the accompanying drawings. FIG. 1 is a schematic diagram to explain a surgical microscope in the present embodiment. In this embodiment, an axial direction of a patient's eye (eye E) is referred to as a Z direction (in a vertical direction in FIG. 1), a horizontal direction (a right-to-left direction of the eye) is referred to as an X direction (in a lateral direction in FIG. 1), and a perpendicular direction is referred to as a Y direction (in a backward and forward direction relative to a drawing sheet of FIG. 1).

A surgical microscope 1 includes a microscopic part 5, an eye characteristic measuring part 7, an imaging optical system 70b, an in-field display system 80, and a controller 100. Each part 5, 7, 70b, and 80 is contained in a casing 3. The microscopic part 5 and the imaging optical system 70b are used as one of observation optical system for observing an eye E during surgery.

The microscopic part 5 is for example a binocular microscope including an optical path PR from the eye E to a right eye of a surgeon and an optical path PL from the eye E to a left eye of the surgeon. Alternatively, the microscopic part 5 may be a monocular microscope. The microscopic part 5 has an objective lens 10 placed in a common optical path between the optical path PR and the optical path PL. In addition, in each of the optical path PR and the optical path PL, a lens 12R or 12L, a lens 14R or 14L, and an eyepiece lens 18R or 18L are arranged. A set of the lens 12R and the lens 14R and a set of the lens 12L and the lens 14L are moved respectively in an optical axis direction by a first drive part 90, thereby forming a pair of zoom systems 16R and 16L. Visible light sources 77 emit visible light for illuminating an anterior segment. The casing 3 is movable in the X, Y, and Z directions with an arm not shown to adjust the position of the casing 3 so that a focal position of the objective lens 10 comes to the anterior segment.

The illumination light emitted from the visible light sources 77 illuminates the anterior segment Ea from front. Reflection light obtained by reflection of the illumination light from the anterior segment passes through the objective lens 10 and a beam combiner 40. Then, the light passing along the optical path PR reaches the eyepiece lens 18R via the zoom system 16R and a beam splitter 71. On the other hand, the light passing along the optical path PL reaches the left eye of the surgeon via the zoom system 16L, the beam combiner 40, and the eyepiece lens 18L. Accordingly, the surgeon can observe an image of the anterior segment by looking through the eyepiece lenses 18R and 18L with his/her right and left eyes.

The eye characteristic measuring part 7 is configured to measure at least one of eye refractive power, corneal shape, and axial length. For instance, the eye characteristic measuring part 7 includes a first measuring optical system (an eye refractive power measuring part) 30, a second measuring optical system (an axial length measuring part) 50, and a third measuring optical system (a corneal shape measuring part) 70.

The second measuring optical system 50 is configured to measure for example the axial length of the patient's eye placed on a surgical position. The second measuring optical system 50 includes for example a light interferometer to measure the axial length after a crystalline lens of the patient's eye is removed. The third measuring optical system 70 is configured to measure for example the corneal shape of the eye E placed in the surgical position.

<Eye Refractive Measuring Optical System>

The first measuring optical system 30 is an optical system for objectively measuring eye refractive power. The first measuring optical system 30 is arranged to project a measuring index onto a fundus Ef and receive reflection light therefrom. The refractive power of the eye E is thus measured based on that light receiving signal.

The first measuring optical system 30 includes for example a light projecting optical system 30a and a light receiving optical system 30b. The light projecting optical system 30a projects a spot index onto the fundus Ef via the center of a pupil Pc. The light receiving optical system 30b takes out a ring-shaped light, through a peripheral portion of the pupil, from the reflection light obtained by reflection of the spot index from the fundus, and forms an image of the ring-shaped reflection light on an imaging element 46.

The light projecting optical system 30a includes a light source 32, a projection lens 34, a hole mirror 36, a dichroic mirror 38, the beam combiner 40, and the objective lens 10. The light source 32 is placed at a position approximately conjugate with the fundus Ef and an aperture of the hole mirror 36 is placed at a position approximately conjugate with the pupil of the eye E.

The light receiving optical system 30b uses the optical path from the objective lens 10 to the hole mirror 36 in common with the light projecting optical system 30a. The light receiving optical system 30b further includes a collimator lens 42, a ring lens 44, and an imaging element (e.g., a two-dimensional imaging element such as CCD and CMOS) 46. The imaging element 46 is located at a position approximately conjugate with the fundus Ef via the objective lens 10, the lens 42, and the ring lens 44. The ring lens 44 consists of a lens part formed of an annular cylindrical lens and a light-shielding part having an annular opening of the same size as the lens part. This ring lens 44 is placed at a position approximately conjugate with the pupil of the eye E. An output signal from the imaging element 46 is transmitted to the controller 100. The dichroic mirror 38 has the property of reflecting light for measuring eye refractive power but transmitting light for measuring axial length.

Measurement light emitted from the light source 32 passes through the pupil center Pc via the projection lens 34, the hole mirror 36, the dichroic mirror 38, the beam combiner 40, and the objective lens 10, and is projected on the fundus Ef.

Reflection light from the fundus passes along the optical path from the objective lens 10 to the hole mirror 36 and is reflected by a reflection surface of the hole mirror 36. The fundus reflection light is then converted into an almost parallel light beam (in the case of an emmetropic eye) by the collimator lens 42. This parallel light beam is taken out as a ring-shaped beam by the ring lens 44 and then received as a ring image by the imaging element 46.

An output signal from the imaging element 46 is stored as image data (measurement image) in a memory 102. Subsequently, the controller 100 detects the image position in respect to each meridian direction based on the images stored in the memory 102 and then performs elliptic approximation by using a least squares method and the like. The controller 100 determines a refractive error in each meridian direction from the approximated elliptic shape and, based on this, measures an eye refractive value (S: spherical power, C: cylinder power, A: astigmatic axis angle). Obtained information on the eye refractive power is stored in the memory 102.

As well as the above configuration, other various methods are adoptable; e.g., a method achieved by projecting a ring index on the fundus Ef through the peripheral portion of the pupil and taking out reflection light through the pupil center so that a ring image is received on the imaging element, and a phase difference method achieved by projecting slit light on the fundus. It is to be noted that the first measuring optical system 30 may also be an eye aberrometor arranged to project measurement light on the fundus Ef and detect the measurement light reflected from the fundus by a wavefront sensor.

<Axial Length Measuring Optical System>

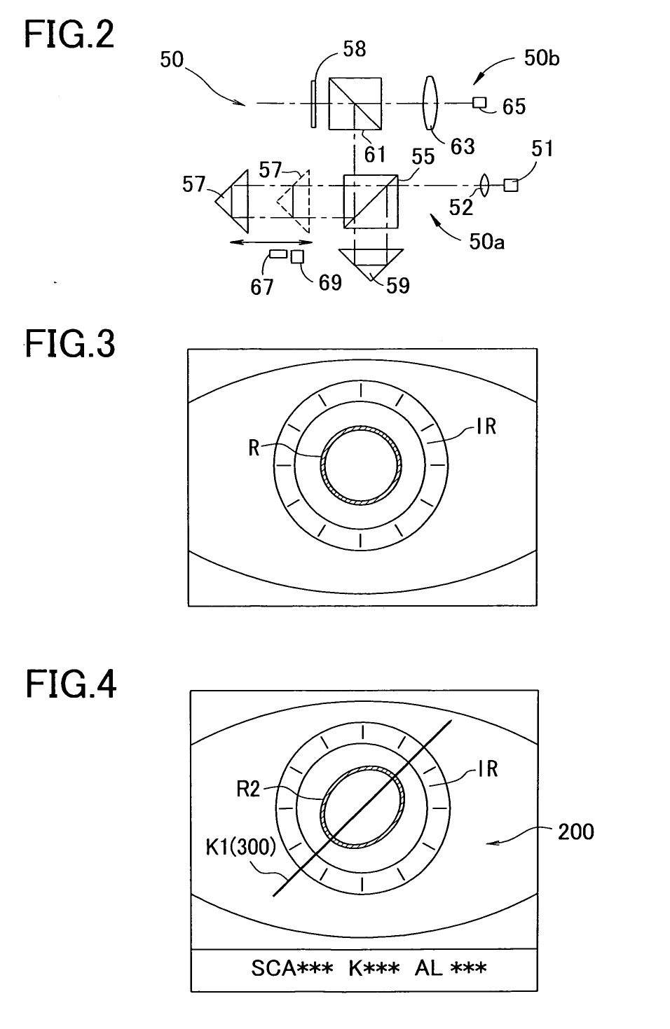

FIG. 2 is a schematic optical diagram to explain the second measuring optical system. The second measuring optical system 50 is an optical system for measuring the axial length by use of an optical coherence tomography. The axial length measuring optical system 50 includes a light projecting optical system 50a and a light receiving optical system 50b. The light projecting optical system 50a is arranged to split light emitted from a measurement light source 51 and project one of the split light beams toward the fundus. The light receiving optical system 50b is arranged to synthesize the one beam reflected from the fundus with the other beam to guide this synthesized interference light to a light receiving element. In an optical path of at least one of the light projecting optical system 50a and the light receiving optical system 50b, an optical member placed movably in an optical axis direction (e.g., a first triangular prism 57) is provided to adjust an optical path difference between the one beam and the other beam.

For instance, the light projecting optical system 50a includes the measurement light source 51 (in this embodiment, also used as a fixation lamp), a collimator lens 52, a beam splitter 55, the first triangular prism (a corner cube) 57, a second triangular prism 59, a polarizing beam splitter 61, a ¼ wave plate 58, the dichroic mirror 38, the beam combiner 40, and the objective lens 10.

The measurement light source 51 emits low coherent light. The collimator lens 52 converts the light emitted from the light source 51 into parallel light. The beam splitter 55 splits the light emitted from the light source 51. The first triangular prism (corner cube) 57 is placed in a transmitting direction of the beam splitter 55. The second triangular prism 59 is located in a reflecting direction of the beam splitter 55.

For instance, the light receiving optical system 50b includes the objective lens 10, the beam combiner 40, the dichroic mirror 38, the ¼ wave plate 58, the polarizing beam splitter 61, a condensing lens 63, and a light receiving element 65.

The light (linearly-polarized light) emitted from the light source 51 is collimated by the collimator lens 52 and then split by the beam splitter 55 into a first measurement beam and a second measurement beam. The split beams are then reflected respectively by the triangular prism 57 (the first measurement beam) and the triangular prism 59 (the second measurement beam) and then returned to be synthesized by the beam splitter 55.

The synthesized beam is reflected by the polarizing beam splitter 61 and converted into a circular polarized beam by the ¼ wave plate 58. The converted light is then irradiated to a cornea and the fundus via the dichroic mirror 38, the beam combiner 40, and the objective lens 10. At that time, when reflected by the cornea and the fundus, the measurement light is converted in phase by ½ wave.

The corneal reflection light and the fundus reflection light pass through the polarizing beam splitter 61 via the objective lens 10, the beam combiner 40, the dichroic mirror 38, and the ¼ wave plate 58. Subsequently, the reflection light passing though the polarizing beam splitter 61 is condensed by the condensing lens 63 and then received by the light receiving element 65.

The triangular prism 57 is used as an optical path length changing member to change an optical path length and is moved linearly in the optical axis direction by driving of the drive part 67 (e.g., a motor) relative to the beam splitter 55. In this case, the optical path length changing member may be a triangular mirror. Further, the position of the moved prism 57 is detected by a position detecting sensor 69 (e.g., a potentiometer, an encoder, etc.).

Although the above explanation shows the configuration that makes the corneal reflection light and the fundus reflection light interfere with each other, the invention is not limited thereto. The invention may be applied to an axial length measuring apparatus provided with an interference optical system including: a beam splitter for splitting light emitted from a light source; a sample arm; a reference arm; and a light receiving element for receiving interference light, in which the light receiving element receives interference light of the measurement light irradiated to a patient's eye via the sample arm and the reference light from the reference arm. In this case, an optical path length changing member is placed in at least one of the sample arm and the reference arm.

In the above configuration, the optical path length of the reference light is changed by linearly moving the prism 57. The invention is not limited thereto and may be applied to a configuration that the optical path length of the reference light is changed by a light delay mechanism using a rotary reflector (e.g., see JP 2005-160694 A).

When a trigger signal to start measurement is output automatically or manually, the controller 100 turns on the measurement light source 51 to irradiate the measurement light to the eye through the light projecting optical system 50a. The controller 100 causes the light receiving element 65 to receive the reflection light obtained by reflection of the measurement light from the eye.

The controller 100 controls driving of the drive part 67 to reciprocate the first triangular prism 57. At the timing when the light receiving element 65 detects the interference light, the controller 100 calculates an axial length based on the received signal output from the light receiving element 65. The obtained axial length information is stored in the memory 102.

In the case where the surgical microscope 1 includes the first measuring optical system 30 and the third measuring optical system 70, when the axial length is to be measured after a crystalline lens is removed, the controller 100 may be configured to indirectly calculate the axial length based on the obtained eye refractive power and corneal shape.

<Corneal Shape Measuring Optical System and Imaging Optical System>

The third measuring optical system 70 includes a light projecting optical system 70a and an imaging optical system 70b (see FIG. 1). For instance, the light projecting optical system 70a has projection light sources 76 and is arranged to project a measurement index on the cornea Ec to measure the corneal shape (curvature, astigmatic axis angle, etc.). The light projecting optical system 70a projects a ring index, for example. Furthermore, it is only necessary to arrange at least three or more point sources on the same circumference centered at the optical axis L1. A light source for an intermittent ring-shaped light may also be adopted. Also, a Placido index projecting optical system for projecting a plurality of ring indices may be adopted. The light sources 76 used herein are for example light sources (LEDs, SLDs, etc.) each of which emits infrared light or visible light. In the following explanation, the light projecting optical system 70a is also used to illuminate the anterior segment by infrared light.

For instance, the imaging optical system 70b includes the objective lens 10, the zoom system 16R, the dichroic mirror 71, an imaging lens 72, and a two-dimensional imaging element 74. This two-dimensional imaging element 74 is placed at a position approximately conjugate with the anterior segment. The dichroic mirror 71 has the property of reflecting infrared light and transmitting visible light.

The imaging optical system 70b images a corneal reflection image obtained by reflection of the measurement light emitted from the light sourced 76 and reflected from the cornea. In the present embodiment, furthermore, the imaging optical system 70b images an image of the anterior segment Ea of the eye E illuminated by the light projecting optical system 70a.

FIG. 3 is a diagram showing the anterior segment image obtained by the anterior segment observation system. The controller 100 obtains the anterior segment image including the ring index R based on the imaging signal output from the imaging element 74 and stores the image in the memory 102.

The controller 100 calculates the corneal shape (e.g., corneal curvature in a strong principal meridian direction and a weak principal meridian direction) based on the ring index image R in the anterior segment image stored in the memory 102 and stores a measurement result in the memory 102. In the case of a corneal astigmatic eye, the index image R has an elliptic shape. The controller 100 therefore determines the astigmatic axis angle by detecting the major axis direction and the minor axis direction.

The third measuring optical system 70 may also be provided as a corneal topography measuring device arranged to project measurement light on a cornea and receive the measurement light reflected from the cornea to measure corneal topography.

The controller 100 may use the imaging optical system 70b as an eye tracker to detect movement information of the eye E. For instance, the controller 100 detects at least one of a rotation state of the eye and a positional displacement in the X and Y directions by image processing based on the imaging signal output from the imaging element 74. For instance, the movement of the eye E is detected by extracting characteristic points (e.g., an outline of an iris IR) of the eye E and comparing them between continually obtained images. As the method of detecting positional displacement between two images, various image processing methods (a method using various correlation functions, a method utilizing a Fourier transform, and a method using matching of characteristic points) may be used. The eye rotation information is detected by for example obtaining the rotation direction and the rotation amount of characteristic portions such as blood vessels, iris, or others in the anterior segment image, by image processing.

<In-Field Display System 80>

The in-field display system 80 is provided to display the information on surgery within the field of the microscopic part 5. The in-field display system 80 includes a display part 82, a projection lens (a projection lens system) 84, and a half mirror 86. The display part 82 used herein is a display device such as an LCD, an organic EL, a liquid crystal projector, etc. The half mirror 86 is placed in the optical path PL to synthesize reflection light from the eye E illuminated by the anterior segment illumination light and projection light from the display part 82.

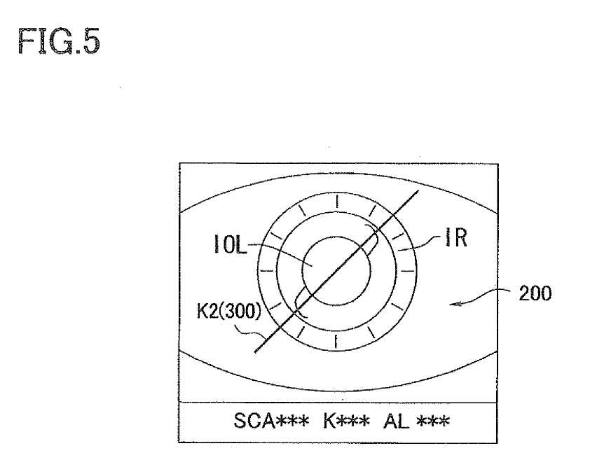

FIGS. 4 and 5 are examples of observation images through the eyepiece lens 18L, showing an example of a state where a microscopic image and an indication image are superimposed one on the other. While looking through the microscopic part 5, the surgeon views a superimposed state of a microscopic image 200 directly observed through the optical path PL and an indication image 300 electronically displayed on the display part 82.

The controller 100 causes the display part 82 to display a measurement result obtained by the eye characteristic measuring part 7. For instance, the controller 100 displays, on a screen of the display part 82, eye refractive power SCA, corneal shape K, and axial length AL as the measurement result. Thus, the examiner can confirm the characteristics of the eye E as well as the microscopic image and also can utilize this measurement result in the surgery.

The controller 100 outputs guide information to the in-field display system 80 to guide a surgery with an IOL (IOL surgery) based on the measurement result obtained by the eye characteristic measuring part 7. For instance, the controller 100 generates a guide pattern and superimposes the guide pattern on the observation image obtained by the microscopic part 5. For instance, the guide pattern is electronically displayed on the display part 82 and presented together with the observation image to the surgeon's eye via the half mirror 86.

For instance, the controller 100 generates a graphic representing astigmatism information of the eye based on the astigmatic axis information obtained by the eye characteristic measuring part 7 and then displays the graphic by superimposing it on the observation image to be observed through the microscopic part 5. The corneal shape of the eye E before incision placed in the surgical position is measured by the eye characteristic measuring part 7. The controller 100 generates a graphic representing the astigmatic axis information based on the astigmatic axis information measured before incision. Further, the corneal shape of the eye E placed in the surgical position after insertion of the IOL is measured by the eye characteristic measuring part 7. The controller 100 generates a graphic representing the astigmatic axis information based on the astigmatic axis information measured after insertion of the IOL.

For a guide to be superimposed on the anterior segment image, for example, a guide pattern (see a line K1 in FIG. 4 and a line K2 in FIG. 5) is displayed in a direction corresponding to the strong meridian direction of the eye E. Further, when a guide pattern representing the shape of an IOL is used as a guide, the arrangement position of the IOL can be easily adjusted. Preferably, the controller 100 corrects the generation angle of the guide pattern based on the rotation information of the eye obtained from the eye characteristic measuring part 7. It is to be noted that the controller 100 can select whether or not to display the guide pattern (e.g., based on an operation signal from the operating part 104).

The controller 100 controls the eye characteristic measuring part 7 to obtain in advance the characteristics of the eye E before a substance is injected into the eye E and then displays the obtained characteristics to enable comparison with the characteristics of the eye E obtained by the eye characteristic measuring part 7. Accordingly, the eye characteristics before injection of the substance and the eye characteristics after injection of the substance are compared.

The in-field display system 80 may be a head-mounted display. In this case, the controller 100 displays the information on surgery in a superimposed position on the anterior segment image obtained by the imaging element 74.

Further, the in-field display system 80 has only to display the information on surgery within the visual field of the surgeon who is looking through the microscopic part 5. For instance, the in-field display system 80 may be provided as a projection optical system for optically projecting a guide pattern image on the anterior segment. Herein, the pattern image projected on the anterior segment is reflected by the anterior segment and then passes along the observation optical path PL and is presented together with the observation image to the surgeon's eye.

<Control System>

The controller 100 makes controls of the entire apparatus, calculations of the eye characteristics, calculations of the IOL power, and others. The controller 100 is connected to the first drive part 90, the visible light sources 77, the light source 32, the imaging element 46, the light source 51, the light receiving element 65, the drive part 67, the position detecting sensor 69, the projecting light sources 76, the imaging element 74, the display part 82, the memory 102, the operating part (control part) 104, an external display 106, and an input device 108.

The memory 102 is used as a storage part. The operating part 104 is operated manually and for example provided with a switch 104a for enabling manual input of a trigger to start measurement to each measuring optical system of the eye characteristic measuring part 7. In this case, it may be arranged that switches are placed in correspondence with the measuring optical systems individually or that a single switch is provided to operate a plurality of measuring optical systems. The switch 104a is preferably placed in a position (e.g., the casing 3) accessible by a hand of the surgeon who is looking through the microscopic part 5.

The controller 100 displays on a screen of the external display 106 at least one of the anterior segment image imaged by the imaging element 74, each measurement result measured by the eye characteristic measuring part 7, and other surgical information. Other surgical information may include induced astigmatism information, the characteristics (eye refractive value, corneal curvature, axial length, etc.) of the eye measured in a sitting position, and others. Other surgical information is input in the controller 100 through the input device 108. The controller 100 may be configured to cause the display part 82 to display each surgical information appearing on the screen of the external display 106.

The procedures of the IOL surgery are briefly explained below. As advance preparation, the surgeon makes the examiner sit in a chair and obtains the information on IOL surgery from the eye E (e.g., corneal shape, eye refractive power, axial length, etc.). Based on the obtained information on the eye E, an IOL to be inserted in the eye is determined.

The device for measuring the eye refractive power in advance is selected from an autorefractometer, an eye aberrometer (a wavefront sensor), and others, for example. The device for measuring the corneal shape is selected from an autokeratometer, a corneal topography measuring device, and others. The device for measuring the axial length is for example a device using light or ultrasonic wave.

When actual surgery is to be conducted, the surgeon makes the patient lie supine on a surgical table so that the eye E is placed in the surgical position. The surgeon performs the surgery while looking through the surgical microscope. The surgeon first incises the eye E and then injects a viscoelastic substance into the eye to protect the tissues of the eye E. Subsequently, the surgeon fragments an opaque nucleus of a crystalline lens by use of a cataract surgery device and extracts the fragmented nucleus.

The surgeon then supplies the viscoelastic substance in the eye and inserts the IOL in a lens capsule by use of an injector. The surgeon adjusts the position of the IOL with forceps. In the case of a toric lens, the angle of the IOL is adjusted so as to correct astigmatism of the eye E. After completion of adjustment of the IOL, the surgeon removes the viscoelastic substance and instead injects physiological saline into the eye. The surgeon then stitches an incision.

The cataract surgery device is generally a device including a hand piece to ultrasonically emulsify and remove the nucleus. In recent years, there is also proposed a device using a femtosecond laser to fragment the nucleus of a crystalline lens.

The surgical microscope in the present embodiment including the eye characteristic measuring part 7 and the imaging optical system 70b can obtain the eye characteristics and the image of the eye E of the patient in a supine position. For instance, the controller 100 controls each measuring optical system of the measuring part 7 to continually measure the refractive power, corneal shape, and axial length of the eye, and records the obtained measurement results in the memory 102. The controller 100 controls the imaging optical system 70b to continually image the eye E, and records imaged images in the memory 102. At that time, the controller 100 associates the measurement results and the imaged images obtained at the same timings and records them.

Further, the controller 100 displays the obtained measurement result on the display part 82 and superimposes it on the image of the eye E. The controller 100 also may be configured to display the obtained measurement result and the imaged image on the screen of the external display 106.

The controller 100 also may be configured to associate each step of the surgery with each measurement result. For instance, the controller 100 stores a measurement value or values obtained in at least one of the steps of: before incision; after incision; injection of the first viscoelastic substance; extraction of the crystalline lens; injection of the second viscoelastic substance; insertion of the IOL; positioning of the IOL; injection of physiological saline; and completion of the surgery.

The controller 100 controls the eye characteristic measuring part 7 based on the automatically generated trigger signal to measure the eye E. For instance, the controller 100 may be configured to obtain the characteristics of the eye E over time (e.g., continuously or at predetermined time intervals). The controller 100 may be arranged to activate the eye characteristic measuring part 7 in response to the operation signal output by the switch 104a when manually operated, and sequentially obtain the characteristics of the eye E at each time.

<Measurement Before Incision>

The surgical microscope in the present embodiment can effectively assist the above IOL surgery. As a first method, for example, the controller 100 controls the measuring part 7 based on the trigger signal to obtain the characteristics of the eye E after the eye is placed in the surgical position but before incision.

FIG. 4 shows an example of an observation image through the eyepiece lens 18L, showing an example of a state where the microscopic image before incision and the indication image are superimposed one on the other. The controller 100 for example measures the astigmatic axis angle of the eye E before incision by use of the third measuring optical system 70 and outputs the information on the calculated axis angle to the display part 82. The calculated axis angle information is used as e.g. a guide to place the toric IOL in the eye. The controller 100 displays an astigmatic axis index (see the line K1 in FIG. 4) representing the corneal astigmatic axis direction by use of the above detection result in the astigmatic axis direction.

The controller 100 for example determines the display angle of the line K1 corresponding to the astigmatic axis angle calculated as above and then synthesizes the line K1 so as to pass through the center of a ring index R2 (the optical axis of the in-field display system 80). The astigmatic axis index (K1) is displayed to indicate the corneal strong meridian direction of the eye E, for example. The shape of the index is not limited to a line. For example, the controller 100 may cause the display part 82 to display a graphic representing an IOL and adjust the display angle of the graphic according to the corneal astigmatic axis of the eye E.

The controller 100 displays the astigmatic axis index (K1) on the display part 82 during surgery after incision of the eye. In particular, it is preferable to display the index at the time of insertion of the IOL and at the time of positioning of the IOL. Thus, the surgeon can easily make positioning of the toric IOL. Even when the astigmatic axis is displaced between the supine position and the sitting position, the surgeon can place the IOL in a proper position. Further, it is possible to avoid any influence of the eye rotation caused when the patient moves from the sitting position to the supine position.

Furthermore, the controller 100 may be configured to correct the display angle of the astigmatic axis index (K1) based on the information obtained as above on movement of the eye E. For instance, the controller 100 performs image processing to compare the anterior segment image obtained when the corneal shape is measured in the supine position and the anterior segment image continually obtained during surgery and, based on a comparison result, detects the rotation information of the eye E. The controller 100 then corrects the display angle of the astigmatic axis index (K1) based on the detected eye rotation information. The controller 100 may also be configured to detect movement of the eye E in the X and Y directions and, based on this detection result, correct an appearance position of the index (K1) in the X and Y directions.

The controller 100 may calculate the IOL power by substituting the characteristics of the eye E before incision (and in the supine position) obtained as above into an IOL calculating formula. In this case, the controller 100 may combine both measurement values in the supine position and the sitting position. Further, the controller 100 may calculate the IOL power based on the corneal curvature and the axial length before incision (and in the supine position).

In the above manner, the IOL power is calculated based on the measurement values obtained according to a patient's posture when the IOL is to be inserted in his/her eye. Thus, appropriate astigmatism correction can be achieved. For example, even in the case where the corneal curvature is different between the supine position and the sitting position, the surgeon can calculate an appropriate IOL value.

The controller 100 further obtains, through the input device 108, the characteristics of the eye E obtained when the patient is in the sitting position. The controller 100 outputs the measurement values (e.g., corneal shape, eye refractive power, and axial length) to the display part 82 to enable comparison of those values between the sitting position and the supine position. As a displaying method, there are conceivably displaying, in parallel, both the measurement values obtained in the sitting position and in the supine position, displaying a displacement between a measurement value obtained in the sitting position and a measurement value obtained in the supine position, and so on. In this way, if a large displacement, or difference, exists between the measurement values obtained in the supine position and in the sitting position, surgery can be performed in consideration of the displacement.

An example of the astigmatic axis angle of a cornea is given below. In the case where a displacement in axis angle is large, the controller 100 determines a placement angle of the toric lens by using the corneal astigmatic axis measured in the supine position. The controller 100 reflects the determined placement angle into the aforementioned guide pattern. In this case, in order to consider the eye rotation resulting from changes in patient's posture, the controller 100 may compare the astigmatic axis angle with reference to the axis angle corresponding to the characteristic point such as a blood vessel of sclera, iris, and a marking. An example of the corneal curvature is given. In the case where a displacement in curvature is large, the controller 100 calculates the IOL power by using the corneal curvature measured in the supine position.

When the IOL power is to be calculated, the controller 100 may calculate the power by combining both the measurement values obtained in the supine position and the sitting position. For instance, the IOL power is calculated based on the corneal curvature obtained in the supine position and the axial length obtained in the sitting position.

<Measurement Until End of Surgery After Start of Incision>

As a second method, the controller 100 obtains the characteristics of the eye E before the end of surgery after the eye E is incised. In the following explanation of the second method, measurement values before incision include measurement values obtained in the sitting position and measurement values obtained in the supine position.

The controller 100 obtains the characteristics of the eye E after the nucleus of a crystalline lens is removed. For instance, the controller 100 measures an axial length of the eye E from which the nucleus has been removed, and calculates the IOL power based on the measured axial length value. After the nucleus is removed, the viscoelastic substance is injected into the eye. The controller 100 preferably calculates the axial length by using an intraocular refractive index determined by considering that a part of the intraocular substance is a viscoelastic substance. The axial length is calculated by an expression of “Scanning distance (ΔZ) of Optical path length changing member placed in an interferometer/Refractive index of human eye”. Thus, the controller 100 obtains a measurement value by using the refractive index determined in consideration of the viscoelastic substance.

The axial length to be obtained in the state where the cataract nucleus is removed is measured by the measurement light not blocked by the cataract nucleus and does not depend on variations in refractive index due to the stage of cataract. Accordingly, a correct axial length value can be obtained and thus the IOL power appropriate to the characteristics of the eye E can be calculated. Thus, the eye E can be properly corrected.

At that time, when the viscoelastic substance is injected in amounts more than necessary, the cornea so expands as to change the corneal shape. This results in a longer axial length.

The controller 100 obtains in advance the characteristics of the eye E before injection of the viscoelastic substance (before incision or after incision). The controller 100 outputs a measurement value before incision and a measurement value continually obtained after incision to the display part 82 so as to enable comparison between those values (e.g., parallel display of both the values or display of a displacement between the values). At that time, the measurement value after injection is sequentially updated and the comparison display is sequentially updated. The measurement values to be obtained may include the corneal shape, the eye refractive power, the axial length, etc.

The controller 100 outputs, for example, the corneal shape before injection and the corneal shape (preferably corneal curvature) during injection of the viscoelastic substance to the display part 82 to enable comparison between those corneal shapes. This enables the surgeon to inject the viscoelastic substance so that the displacement in corneal shape between after injection and before injection falls within a permissible range. In this case, the controller 100 may display the measurements in parallel or display the displacement between the measurement values. Furthermore, the controller 100 may also graphically display the displacement between the measurement values. At that time, the controller 100 may store an injection amount of the viscoelastic substance in the memory 104.

Accordingly, the corneal shape can be adjusted during injection of the viscoelastic substance. This can avoid variations in axial length due to excess and deficiency of the viscoelastic substance. Since a correct axial length value can be obtained as above, an IOL appropriate to the characteristics of the eye E can be prepared.

Moreover, the controller 100 may display the measurement values between before incision and after incision (e.g., parallel display of both measurement values or display of a difference between the measurement values) so as to enable their comparison. The controller 100 obtains in advance the characteristics of the eye E before incision.

At that time, the controller 100 outputs the characteristics of the eye before incision and the characteristics continually obtained after incision to the display part 82 to enable their comparison. At that time, the measurement value after incision is continually updated and the comparison display is continually updated.

In the above manner, an amount of change in measurement value (e.g., corneal shape, eye refractive power, and axial length) in the states before and after incision can be obtained. For instance, when the viscoelastic substance is injected into the eye, the controller 100 obtains the characteristics of the eye E and determines changes in measurement value after the viscoelastic substance is injected.

In the above second method, it may be arranged such that, when the physiological saline is injected in the eye after alignment of the IOL, the controller 100 obtains the characteristics of the eye E and determine a change in measurement value measured after injection of the physiological saline.

For instance, the controller 100 obtains the characteristics of the eye E after the alignment of the IOL. Herein, after completion of alignment of the IOL, the physiological saline is injected. At that time, the controller 100 outputs a measurement value obtained before injection of the viscoelastic substance (before or after incision) and a measurement value continually obtained after start of injection of the physiological saline to the display part 82 so as to enable their comparison (e.g., parallel display of both measurement values or display of a difference between the measurement values). At that time the measurement value after injection is sequentially updated and the comparison display is sequentially updated. The measurement values to be obtained may include the corneal shape, the eye refractive power, the axial length, etc.

For instance, when the physiological saline is injected in amounts more than necessary, the cornea expands, changing the corneal shape. Therefore, the controller 100 outputs the corneal curvature obtained before injection of the viscoelastic substance and the corneal curvature obtained after injection of the physiological saline to the display part 82 so as to enable their comparison. Accordingly, the surgeon is allowed to inject the physiological saline so that the displacement in corneal shape falls within a permissible range.

The controller 100 also may output the axial length before surgery (in the sitting position, before incision, after removal of the nucleus, etc.) and the axial length after injection of the physiological saline to the display part 82 to enable their comparison. Accordingly, the surgeon can avoid injection of an excessive amount of physiological saline. The eye E can therefore be adjusted to have an axial length appropriate to the IOL inserted therein.

<Measurement After Completion of Surgery>

As a third method, for example, the controller 100 obtains the characteristics of the eye E after completion of surgery. Herein, the controller 100 measures eye refractive power as the characteristics of the eye E and outputs a measurement result to the display part 82. Accordingly, it is possible for the surgeon to check whether a desired correction result is achieved by the surgery. In case the result shows insufficient correction, the surgeon can promptly perform further surgery. It is to be noted that “after completion of surgery” may also be defined as “after injection of the physiological saline” or “after completion of stitching”, for example.

The controller 100 displays, as the measurement result, spherical power S, cylinder power C, and astigmatic axis angle A. In the case of using the wavefront sensor, the controller 100 may display the measurement result in the form of a map.

The controller 100 measures the corneal shape as the characteristics of the eye E and outputs the measurement result to the monitor 72. FIG. 5 shows an example of an observation image through the eyepiece lens 18L, showing an example of a state where the microscopic image after IOL insertion and the indication image are superimposed one on the other. For instance, the controller 100 measures the astigmatic axis angle of the eye E after completion of the surgery (e.g., after completion of injection of the physiological saline) by using the third measuring optical system 70, and outputs the information on the calculated axis angle to the display part 82. The calculated axis angle information is utilized as, for example, a guide to check whether or not the toric IOL is appropriate to the corneal astigmatic axis after surgery. The controller 100 displays an astigmatic axis index (see the line K2 in FIG. 5) indicating the corneal astigmatic axis direction by use of the detection result in the above astigmatic axis direction.

The controller 100 may display the measurement value obtained by the measuring part 7 before injection (preferably, before incision) and the measurement value obtained by the measuring part 7 after completion of the surgery to enable comparison between the measurement values (e.g., parallel display of both the measurement values or display of a displacement between the measurement values).

For instance, the controller 100 displays the corneal shape before injection (preferably before incision) and the corneal shape after surgery to enable their comparison. Accordingly, a change in corneal shape by the surgery is measured properly. In this case, the controller 100 may display the corneal astigmatic axis angle before injection (preferably before incision) and the corneal astigmatic axis angle after injection to enable their comparison (e.g., parallel display of both the measurement values or display of a displacement between the measurement values). Accordingly, a change in the corneal astigmatic axis by the surgery can be measured properly. Thus, the surgeon can properly estimate an appropriate placement position of the toric IOL.

For instance, the controller 100 displays the axial length before injection and the axial length after surgery to enable their comparison. Accordingly, a change in axial length by the surgery is measured properly. This makes it possible for the surgeon to properly estimate an appropriate IOL to the eye E.

Furthermore, the controller 100 measures the corneal shape and the axial length as the characteristics of the eye E and outputs the measurement result to the monitor 72. The controller 100 further calculates the IOL power based on the obtained corneal shape and axial length and outputs a calculation result to the monitor 72. Thereafter, the controller 100 displays a first optical characteristic corresponding to the inserted IOL and a second optical characteristic corresponding to the IOL obtained based on the characteristics of the eye E after surgery to enable their comparison (e.g., parallel display of both optical characteristics or display of a difference between the optical characteristics).

As a fourth method, the controller 100 causes the memory 102 to store the eye characteristics on a number of surgically operated eyes, each being obtained from the placement of an eye E in a surgical position to the end of surgery. Based on the obtained measurement results, a data base representing changes in eye characteristics due to the IOL surgeries is created. It is therefore possible to presume a correction result by utilizing the measurement result of the eye E.

For instance, there may be a case where the corneal shape changes between before surgery and during surgery (or after surgery). In this case, by use of the above data base, a first amount of change in corneal shape between before surgery and during surgery is obtained. Also, there may be a case where the axial length changes between before surgery and during surgery (or after surgery). In this case, by use of the above data base, a second amount of change in axial length between before surgery and during surgery is obtained.

In the case where the IOL power is to be detected based on the corneal shape and axial length before surgery, at least one of the first change amount and the second change amount obtained as above is used as a correction parameter. For instance, the controller 100 calculates a presumed corneal shape during surgery by adding the first change amount to the corneal shape before surgery, and further calculates the IOL power by using the calculated presumed corneal shape.

In the above embodiment, an intraocular pressure measuring system may be provided as the eye characteristic measuring part 7. In this case, for example, the intraocular pressure measuring system may be provided with an ultrasonic probe including a transmitting and receiving part for transmitting an ultrasonic beam to a cornea in non-contact relation and receiving a reflection wave of the ultrasonic beam reflected from the cornea. Based on characteristics of the corneal reflection wave received by the probe, intraocular pressure of a patient's eye is measured. The ultrasonic wave is preferable because it less influences an eye to undergo surgery. Not limited thereto, it may be arranged to measure intraocular pressure in non-contact relation by detecting a deformed state of a cornea when a fluid is injected toward the cornea from a nozzle.

When the intraocular pressure under surgery is measured by the intraocular pressure measuring system in the above manner, for example, it is possible to properly adjust the intraocular pressure in injecting physiological saline in the eye.

In a check after insertion of the IOL, as mentioned above, it is possible to confirm whether or not the examinee's eye has returned to a previous state before surgery. Thus, the surgery can be conducted properly.

The surgical microscope is provided with an optometry unit (e.g., the eye characteristic measuring part 7) for monitoring the state of the examinee's eye. The controller 100 obtains first information measured by the optometry unit before injection of the viscoelastic substance into the eye, obtains second information measured by the optometry unit after insertion of the IOL, and compares the first information and the second information to output a comparison result. For instance, the controller 100 may compare the first information and the second information to determine whether or not the state of the examinee's eye has returned to the previous state before surgery, and output a determination result.

Examples of the optometry unit are a corneal shape measuring unit, an intraocular measuring unit, an OCT (optical coherence tomography) unit, and an axial length measuring unit.

In the case of the intraocular measuring unit, the controller 100 obtains a first intraocular pressure measured by the intraocular pressure measuring unit before injection of the viscoelastic substance into the eye, obtains a second intraocular pressure measured by the intraocular pressure measuring unit after insertion of the IOL, and compares the first intraocular pressure and the second intraocular pressure to output a comparison result. The controller 100 then outputs the first intraocular pressure and the second intraocular pressure to an output unit to enable their comparison.

In the OCT (optical coherence tomography) unit to obtain a tomographic image of an anterior segment of an examinee's eye, the controller 100 obtains a first tomographic image measured by the OCT unit before injection of the viscoelastic substance into the eye, obtains a second tomographic image measured by the OCT unit after insertion of the IOL, compares the first tomographic image and the second tomographic image to output a comparison result (e.g., it represents a difference between an anterior segment shape based on the first tomographic image and an anterior segment shape based on the second tomographic image). The controller 100 outputs the first and second tomographic images to an output device to enable their comparison.

In the case of the corneal shape measuring unit, the controller 100 obtains a first corneal shape measured by the corneal shape measuring unit before injection of the viscoelastic substance into the eye, obtains a second corneal shape measured by the corneal shape measuring unit after insertion of the IOL, and compares the first corneal shape and the second corneal shape to output a comparison result. The controller 100 outputs the first and second corneal shapes to an output device to enable their comparison. For instance, the controller 100 compares the first corneal shape (e.g. astigmatic axis angle) and the second corneal shape (e.g., astigmatic axis angle) by considering induced astigmatism caused by incision of the anterior segment, and outputs a comparison result.

The foregoing detailed description has been presented for the purposes of illustration and description. Many modifications and variations are possible in light of the above teaching. It is not intended to be exhaustive or to limit the subject matter described herein to the precise form disclosed. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims appended hereto.

1. An ophthalmic surgical microscope comprising:
an observation optical system for observing a patient's eye during surgery;
a corneal shape measuring unit for measuring a corneal shape of the patient's eye placed in a surgical position; and
a controller configured to output guide information for guiding a surgery with an intraocular lens based on a measurement result obtained by the corneal shape measuring unit to an output device.

2. The ophthalmic surgical microscope according to claim 1, wherein the corneal shape measuring unit measures the corneal shape at least one of before surgery, during surgery, and after surgery.

3. The ophthalmic surgical microscope according to claim 1, wherein the controller obtains a first corneal shape measured by the corneal shape measuring unit before injection of a viscoelastic substance into the eye, obtains a second corneal shape measured by the corneal shape measuring unit after injection of the viscoelastic substance into the eye, and outputs the first corneal shape and the second corneal shape to the output device to enable their comparison.

4. The ophthalmic surgical microscope according to claim 1, wherein the controller obtains a first corneal shape measured by the corneal shape measuring unit before injection of a viscoelastic substance into the eye, obtains a second corneal shape obtained by the corneal shape measuring unit after insertion of the intraocular lens, and outputs the first corneal shape and the second corneal shape to the output device to enable their comparison.

5. The ophthalmic surgical microscope according to claim 1, further including an intraocular pressure measuring unit for measuring an intraocular pressure of the patient's eye placed in the surgical position.

6. The ophthalmic surgical microscope according to claim 5, wherein the controller obtains a first intraocular pressure measured by the intraocular pressure measuring unit before injection of a viscoelastic substance into the eye, obtains a second intraocular pressure obtained by the intraocular pressure measuring unit after insertion of the intraocular lens, and outputs the first intraocular pressure and the second intraocular pressure to the output device to enable their comparison.

7. The ophthalmic surgical microscope according to claim 1, further including an OCT (optical coherence tomography) unit to obtain a tomographic image of an anterior segment of the eye,
wherein the controller obtains a first tomographic image measured by the OCT unit before injection of a viscoelastic substance into the eye, obtains a second tomographic image measured by the OCT unit after insertion of the intraocular lens, and outputs the first tomographic image and the second tomographic image to the output device to enable their comparison.

8. The ophthalmic surgical microscope according to claim 1, further including an optometry unit for monitoring a state of the eye,
wherein the controller obtains a first information obtained by the optometry unit before injection of a viscoelastic substance into the eye, obtains a second information obtained by the optometry unit after insertion of the intraocular lens, and compares the first information and the second information and outputs a comparison result to the output device.

9. The ophthalmic surgical microscope according to claim 1, wherein the controller generates a graphic representing astigmatic axis information based on the astigmatic axis information obtained by the corneal shape measuring unit, and superimposes the graphic on an observation image observed through the observation optical system.

10. The ophthalmic surgical microscope according to claim 1, further including an axial length measuring unit for measuring an axial length of the patient's eye placed in the surgical position.

11. The ophthalmic surgical microscope according to claim 10, wherein the axial length measuring unit includes a light interferometer to measure an axial length of the patient's eye after removal of a crystalline lens.

12. The ophthalmic surgical microscope according to claim 1, wherein the corneal shape measuring unit measures the corneal shape after incision of the patient's eye placed in the surgical position, and
the controller generates a graphic representing astigmatic axis information based on the astigmatic axis information obtained after incision.

13. The ophthalmic surgical microscope according to claim 1, wherein the corneal shape measuring unit measures the corneal shape after insertion of the intraocular lens in the patient's eye placed in the surgical position, and
the controller generates a graphic representing astigmatic axis information based on the astigmatic axis information measured after insertion of the intraocular lens.
